(19) United States
(12) Patent Application Publication
(10) Pub. No.: US 2007/0073089 A1
(43) Pub. Date: Mar. 29, 2007

(54) Process for the Isomerisation of Alkenyl Alkoxybenzenes

(75) Inventors: Walter Kuhn, Holzminden (DE); Hans-Ulrich Funk, Lauenförde (DE); Gerhard Senft, Holzminden (DE); Christoph Dittert, Hoxter (DE); Torsten Stephan, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/534,806

(22) Filed: Sep. 25, 2006

(30) Foreign Application Priority Data
Sep. 26, 2005 (DE) 10 2005 045 945.5

(51) Int. Cl. C07C 43/02; C07C 43/20

(52) U.S. Cl. 568/627

(57) ABSTRACT

A process for the catalytic double bond isomerisation of alkenyl alkoxybenzenes [alkoxy-substituted (2-alkenyl)-benzenes] (with a non-conjugated double bond in the alkenyl group) of formula A 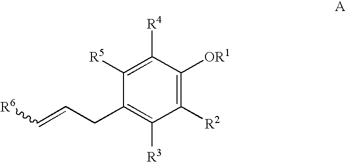
in the presence of a catalytically active quantity of alkali and/or alkaline earth C₁-C₆ alcoholates to the corresponding alkenyl alkoxybenzenes [alkoxy-substituted (1-alkenyl)-benzenes] (with a double bond in the alkenyl group which is conjugated with the benzene ring) of formula B 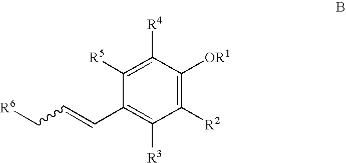 is described

### FIELD OF THE INVENTION

The present invention relates to a process for the catalytic double bond isomerisation (referred to in the following as isomerisation) of alkenyl alkoxybenzenes [alkoxy-substituted (2-alkenyl)-benzenes] (with a non-conjugated double bond in the alkenyl group) of formula A

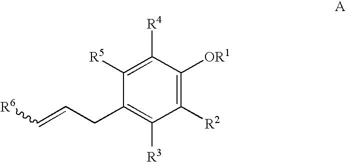

in the presence of a catalytically active quantity of alkali and/or alkaline earth C₁-C₆-alcoholates to the corresponding alkenyl alkoxybenzenes [alkoxy-substituted (1-alkenyl)-benzenes] (with a double bond in the alkenyl group which is conjugated with the benzene ring) of formula B.

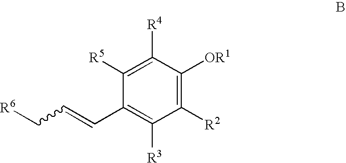

The wavy line in formulae A and B denotes that the double bond can be cis- or trans-configured; for the meaning of radicals R¹ to R⁶ see below.

Compounds of formula B exhibit a larger conjugated system than compounds of formula A and are therefore in principle more stable. Furthermore, compounds of formula B usually exhibit interesting odours. There has therefore for some time been the need to synthesise alkenyl alkoxybenzenes of formula B.

### BACKGROUND

DE 1 936 727 describes the isomerisation of alkenyl aromatic compounds by means of ruthenium or osmium catalysts. According to Helvetica Chimica Acta 1992, 75(5), 1604, hexaaquaruthenium catalysts are used.

DE 2 157 534 proposes as the catalyst for such an isomerisation a mixture of metallic sodium, sodium hydroxide and aluminium oxide.

GB 525705 describes the double bond isomerisation of alkenyl aromatic compounds using alkali metal hydroxides and glycols or polyglycols as diluents for the alkali metal hydroxides. Inter alia, eugenol methyl ether is reacted in the presence of potassium hydroxide, diethylene glycol monoethyl ether and ethanolamine at 160-180° C. Isoeugenol methyl ethers with 95% yield were thereby obtained, wherein the trans double bond isomer was predominant.

Zhurnal Organicheskoi Khimii 1988, 24(6), 1248-1253, examined the kinetics of the isomerisation of allylbenzene (not a compound of formula A) in the presence of lithium tert-butanolate in N,N-dimethylacetamide at 25° C. or in the presence of potassium tert-butanolate in tert.-butanol at 75° C. Using a 0.5 molar solution of the more active catalyst system lithium tert-butanolate in N,N-dimethylacetamide, the rate of isomerisation was apparently measured at up to an approximately 4 molar concentration of allylbenzene. It was found that as the concentration of the allylbenzene rose, the rate of isomerisation became smaller, i.e. the reaction slower. Using a 0.5 molar solution of potassium tert-butanolate in tert.-butanol, the rate of isomerisation was apparently measured at up to approximately 1 molar concentration of allylbenzene. Furthermore, the rate of isomerisation was examined as a function of the catalyst concentration with a 0.1 molar concentration of allylbenzene. The lowest molar catalyst quantity of lithium tert-butanolate in N,N-dimethylacetamide examined was thereby apparently approximately 31 mol %, in the case of potassium tert-butanolate in tert.-butanol over 100 mol %, both based on allylbenzene. The Hammett constants of some allylbenzenes substituted in the ring were determined, in this context inter alia the rate constants of the isomerisation of methyl chavicol (p-methoxyallylbenzene; estragole) was determined. On the whole, there are no reports on the respective yields and the double bond configurations obtained.

The Vietnamese document Tap Chi Hoa Hoc 1997, 35(4), 23-26 (Chemical Abstracts 129:135940) presents results on the isomerisation of methyl chavicol to anethole (1-methoxy-4-(1-propenyl)benzene) in the presence of KOH, potassium tert.-butanolate or KF/Al₂O₃. It is described that the solvent-free execution of the isomerisation leads to a higher rate of reaction, as does the addition of 5% of a phase transfer catalyst. Furthermore, it was found there that irradiation with microwaves drastically shortens the reaction time. The yield was thereby in fact not increased in a single case. A 1:1 molar ratio of potassium tert.-butanolate to methyl chavicol is regarded there as optimum. The best isomerisation results were obtained at 80° C. or 110° C. using an equimolar quantity of potassium tert.-butanolate, wherein after 1 to 1.5 hours' reaction time the yield of anethole was 93%. No comments are made on the double bond geometry of the anethole obtained.

It has been found by means of scientific analyses that cis isomers of compounds of formula B are often more toxic than the trans isomers of these compounds. In particular, cis-anethole is 10 to 20 times more toxic than trans-anethole (cf R. Hänsel, J. Hölzel, “Lehrbuch der pharmazeutischen Biologie”, 1996, pp 162 ff.) and for cis-asarone (β-asarone, 1,2,4-trimethoxy-5-(Z)-1-propenylbenzene), in contrast to trans-asarone (α-asarone), a cancerogenic property has been found in animal tests (http://www.omikron-online.de/cyberchem/aroinfo/asaron a.htm). Furthermore, trans isomers of the compound of formula B in general develop an interesting odour quality. There is therefore a need for a production process for compounds of formula B with a high yield of the respective trans isomer.

### SUMMARY OF THE INVENTION

The present invention was accordingly based on the problem of developing an improved process for the isomerisation of compounds of formula A to the compounds of formula B so that the desired compounds of formula B are obtained with as high as possible a yield and with as high as possible a content of the (in odour terms more valuable) trans double bond isomers. Furthermore, the process should be economical and able to be carried out on an industrial scale, and supply the compounds of formula B of high purity (and good odour) quality.

The problem posed is resolved by a process for the production of a compound of formula B from a compound of formula A 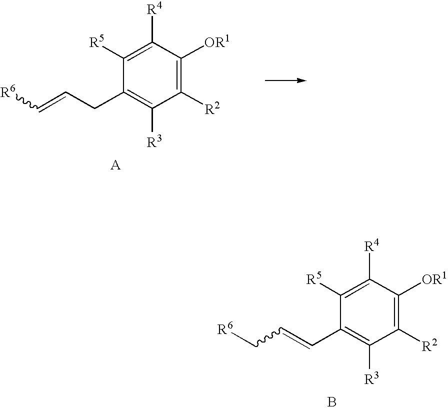
wherein:
R¹ denotes an alkyl radical with 1 to 6 C atoms,
R², R³, R⁴, R⁵ each denote independently of one another hydrogen or a (branched or unbranched) alkyl or alkoxy radical with 1 to 6 C atoms and
R⁶ denotes hydrogen or a (branched or unbranched) alkyl radical with 1 to 6 C atoms;
   or
R¹ and R² are linked together so that an in total 5 to 7-member ring which comprises in total 3 to 6 C atoms and 1 or 2 oxygen atoms (cf the examples below) is formed, and
R³, R⁴, R⁵ each denote independently of one another hydrogen or a (branched or unbranched) alkyl or alkoxy radical with 1 to 6 C atoms, and
R⁶ denotes hydrogen or a (branched or unbranched) alkyl radical with 1 to 6 C atoms,
   in the presence of 0.1 to 10 mol %, of an alkali or alkaline earth alcoholate with 1 to 6 C atoms, based on the quantity used of compound of formula A.

Preferred alkyl radicals with 1-6 C atoms are: methyl, ethyl, n-propyl, isopropyl, butyl, isobutyl, sec.-butyl, tert.-butyl, n-pentyl, isopentyl, tert.-pentyl, n-hexyl, iso-hexyl und tert.-hexyl.

Preferred alkoxy radicals with 1-6 C atoms are: methoxy, ethoxy, propoxy, isopropoxy, butoxy, isobutoxy, tert.-butoxy, pentoxy, isopentoxy, hexoxy and isohexoxy.

It is particularly surprising that the process according to the invention for isomerisation which requires very small quantities of catalyst, with excellent yield supplies compounds of formula B and with very high selectivity leads to the corresponding trans double bond isomers of formula B.

Alkali and/or alkaline earth metal alcoholates with 1 to 6 carbon atoms are used as catalysts in the processes according to the invention. Preferably, apart from these alkali and alkaline earth metal alcoholates, no other, i.e. no additional catalysts active for a double bond isomerisation, are used.

Preferred catalysts are alkali alcoholates, preferably sodium and potassium alcoholates, with 1 to 6 carbon atoms, preferably with 1 to 4 carbon atoms. Apart from this, certain alkaline earth alcoholates are also preferred.

As alkali or alkaline earth metal alcoholates preferably to be used in processes according to the invention can be cited: potassium tert-butanolate, sodium tert-butanolate, lithium tert-butanolate, sodium ethanolate, potassium ethanolate, lithium ethanolate, sodium methanolate, potassium methanolate, lithium methanolate, sodium isopropanolate, potassium isopropanolate, lithium isopropanolate, magnesium ethanolate, magnesium methanolate, calcium ethanolate and calcium methanolate. Furthermore preferred are mixtures thereof.

Particularly preferred are sodium methanolate, sodium ethanolate, sodium isopropanolate, sodium tert-butanolate, potassium methanolate, potassium ethanolate, potassium isopropanolate and potassium tert-butanolate.

Particularly good results are obtained with the catalysts potassium tert-butanolate and sodium tert-butanolate (tertiary alcoholates). An excellent yield, a high (odour) purity and a very high selectivity to the trans double bond isomers of formula B are achieved in particular with potassium tert-butanolate.

In a preferred embodiment according to the invention, the process conditions are set such that a ratio of the trans:cis double bond isomers of formula B is achieved in the range of 80:20 to 99:1, preferably in the range of 90:10 to 98:2 by the isomerisation according to the invention.

The isomerisation according to the invention takes place in the presence of 0.1 to 10 mol % (corresponding to 0.001 to 0.1 molar equivalents) of the said catalyst alcoholates; 0.5 to 8 mol % is preferred, 1 to 5 mol % particularly preferred. The molar equivalents hereby refer to the quantity used of compound of formula A to be isomerised.

Preferred compounds of formula A or of formula B are those in which
R¹ denotes an alkyl radical with 1 to 6 C atoms,
R², R³, R⁴, R⁵ each denote independently of one another hydrogen or methoxy, provided that at least one of radicals R², R³, R⁴, R⁵ is methoxy and
R⁶ denotes hydrogen or methyl;
   or
R¹ und R² are linked together so that an in total 5 or 6-member ring which comprises in total 3 or 4 C atoms and 1 or 2 oxygen atoms is formed, and
R³, R⁴, R⁵ each denote independently of one another hydrogen or methoxy and
R⁶ denotes hydrogen or methyl.

Here for example the isomerisation of methyl chavicol to anethole and of 2,4-dimethoxy-1-allylbenzene to 2,4-dimethoxy-1-propenylbenzene can be cited.

Particularly preferred compounds of formula A are those in which R⁶ denotes hydrogen and R² has the meaning OR² according to formula A1 below, 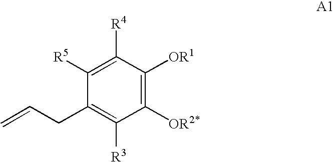
in which:
R¹ denotes an alkyl radical with 1 to 6 C atoms;
R^{2*} denotes methyl;
R³, R⁴ and R⁵ each denote independently of one another hydrogen or methoxy, provided that at least one radical R³, R⁴ or R⁵ denotes hydrogen,
   or
R¹ and R² are linked together so that an in total 5 or 6-member ring which comprises in total 3 or 4 C atoms and 2 oxygen atoms is formed;
R³, R⁴ and R⁵ each denote independently of one another hydrogen or methoxy, provided that at least one radical R³, R⁴ or R⁵ denotes hydrogen.

Compounds of formula A to be used especially preferably in the processes according to the invention (isomerisations) are those of formulae A2 and A3: 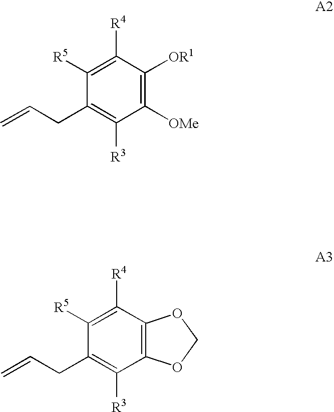
wherein for A2 and A3:
R³, R⁴ and R⁵ each denote independently of one another hydrogen or methoxy, provided that at least one radical R³, R⁴ or R⁵ denotes hydrogen,
   and in addition in formula A2:
R¹ denotes an alkyl radical with 1 to 6 C atoms.

A compound of formula A3 is a compound of formula A in which R¹ and R² are linked together so that an in total 5-member ring which comprises in total 3 C atoms and 2 oxygen atoms is formed.

Compounds of formula A2 to be used especially preferably in the processes according to the invention (isomerisations) are: methyl eugenol (eugenol methyl ether, R¹=methyl, R³, R⁴ and R⁵═H), ethyl eugenol (R¹=ethyl, R³, R⁴ and R⁵═H), iso-amyl eugenol (R¹=iso-amyl, R³, R⁴ and R⁵═H), and elemicin (R¹=methyl, R⁴=methoxy, R³ and R⁵═H).

Compounds of formula A3 to be used especially preferably in the process according to the invention are: safrole (R³, R⁴ and R⁵═H), myristicin (R⁴=methoxy, R³ and R⁵═H), apiole (R³ and R⁴=methoxy, R⁵═H) and dillapiole (R⁴ and R⁵=methoxy, R³═H).

Especially preferred compounds of formula B according to the isomerisation according to the invention are accordingly: methyl isoeugenol (isoeugenol methyl ether), ethyl isoeugenol, iso-amyl isoeugenol, isoelemicin, isosafrole, ilsomyristicin, isoapiole and dillisoapiole.

The reaction temperature for carrying out the processes according to the invention (isomerisations) is preferably in the range of 40 to 190° C., preferably in the range of 60 to 160° C. Provided the preferred tertiary alkali alcoholates, such as for example potassium tert-butanolate, are used as catalysts, temperatures in the range of 60 to 100° C. are preferred.

Isomerisation can be carried out according to the invention in an inert diluent; preferably isomerisation is carried out diluent-free.

If the isomerisation according to the invention is carried out in an inert diluent, (saturated) hydrocarbons, preferably with 10 to 16 carbon atoms, such as for example dodecane, or ethers, in particular those with a boiling point above the reaction temperature, preferably diphenyl ethers, are preferred. Furthermore, the product of formula B to be formed in isomerisation can also be used as diluent. Preferably no glycol or polyglycol is used as diluent. Furthermore, preferably no N,N-dimethylacetamide, no tert.-butanol and no DMSO is used as diluent.

Isomerisation is carried out according to the invention preferably in the absence of a phase transfer catalyst and in the absence of a microwave irradiation.

Isomerisation can be carried out according to the invention continuously or discontinuously. The compounds of formula A used can either be filled into the reaction vessel from the beginning together with the catalyst or, depending on the course of the reaction, be introduced into the reaction vessel continuously or in batches.

### EXAMPLES

The course of the reaction or the progression of the isomerisation according to the invention can be tracked for example by means of gas chromatography. Since the reaction proceeds with high trans-selectivity, the end products occur after washing out the catalyst in often sufficiently high purity. A further purification can take place for example by distillation.

The following examples illustrate the invention. Unless otherwise stated, all data refer to the weight.

### Example 1

### Isomerisation of Eugenol Methyl Ether (Methyl Eugenol) to Isoeugenol Methyl Ether (Methyl Isoeugenol) by Means of Sodium Methanolate

178 g eugenol methyl ether (1 mol) and 1.1 g sodium methanolate (0.02 mol) were stirred at 130° C. over a period of 4 hours. It was then washed with 4 g 10% NaCl solution. The organic phase (171 g) consisted substantially of 81.1% trans-isoeugenol methyl ether, 18.1% cis-isoeugenol methyl ether and 0.6% eugenol methyl ether. The product distilled at 123° C. and 10 mbar vacuum at the top. There remained 12 g residue.

### Example 2

### Isomerisation of Eugenol Methyl Ether (Methyl Eugenol) to Isoeugenol Methyl Ether (Methyl Isoeugenol) by Means of Potassium Tert.-butanolate

178 g eugenol methyl ether (1 mol) and 2 g potassium tertiary butanolate (0.018 mol) were stirred at 70-75° C. over a period of 6 hours. It was then washed with 20 g 15% NaCl solution. The organic phase (175 g) consisted substantially of 95.7% trans-isoeugenol methyl ether, 3.9% cis-isoeugenol methyl ether and 0.1% eugenol methyl ether. The product distilled at 123° C. and 10 mbar vacuum at the top and exhibited a good odour quality. There remained 10 g residue.

### Example 3

### Isomerisation of Eugenol Methyl Ether to Isoeugenol Methyl Ether by Means of KOH (not According to the Invention)

178 g eugenol methyl ether (1 mol) and 3.6 g potassium hydroxide (0.064 mol) were stirred at 120° C. over a period of 9 hours. It was then washed with 20 g 15% NaCl solution. The organic phase consisted substantially of 71.9% trans-isoeugenol methyl ether 16.7% cis-isoeugenol methyl ether and 11.1% eugenol methyl ether.

1. Process for the production of a compound of formula B by catalytic isomerization from an alkenyl alkoxybenzene compound of formula A 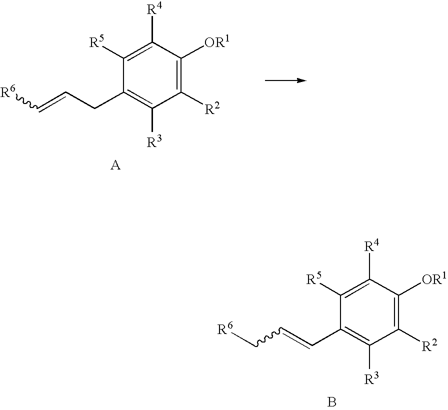
wherein:
R¹ denotes an alkyl radical with 1 to 6 C atoms,
R², R³, R⁴, R⁵ each denote independently of one another hydrogen or an alkyl or alkoxy radical with 1 to 6 C atoms, and
R⁶ denotes hydrogen or an alkyl radical with 1 to 6 C atoms,
wherein
R¹ and R² may be linked together so that an in total 5 to 7-member ring which comprises in total 3 to 6 C atoms and 1 or 2 oxygen atoms is formed, and
said process comprising the step of isomerising said alkenyl alkoxy benzene according to formula A under isomerization conditions in the presence of a catalytically effective amount within the range of 0.1 to 10 mol % of an alkali or alkaline earth alcoholate with 1 to 6 C atoms, based on the quantity used of compound of formula A.

2. Process according to claim 1, wherein isomerisation step is carried out diluent-free.

3. Process according to claim 1, wherein the isomerisation step is carried out in the presence of 0.5 to 8 mol % of the alkali or alkaline earth alcoholate with 1 to 6 C atoms, based on the quantity of the compound of formula A used.

4. Process according to claim 1, wherein the alkali or alkaline earth alcoholate is a sodium or potassium alcoholate with 1 to 4 C atoms.

5. Process according to claim 1, wherein the compound of formula A is a compound according to formula A1 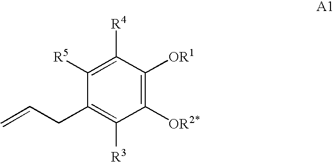
wherein:
R¹ denotes an alkyl radical with 1 to 6 C atoms,
R^{2*} denotes methyl;
R³, R⁴ and R⁵ each denote independently of one another hydrogen or methoxy, provided that at least one radical R³, R⁴ or R⁵ denotes hydrogen,
wherein
R¹ and R^{2*} can be linked together so that an in total 5 or 6-member ring which comprises in total 3 or 4 C atoms and 2 oxygen atoms is formed.

6. Process according to claim 1, wherein the compound of formula A is selected from the group consisting of methyl eugenol, ethyl eugenol, iso-amyl eugenol, elemicin, safrole, myristicin, apiole und dillapiole.

7. Process according to claim 1, wherein the trans:cis ratio of the double bond isomers of formula B is in the range of 80:20 to 99:1, preferably in the range of 90:10 to 98:2.
